(19) United States
(12) Patent Application Publication
(10) Pub. No.: US 2017/0231543 A1
(43) Pub. Date: Aug. 17, 2017

(54) MEDICAL BODILY FLUID SAMPLING DEVICE

(71) Applicant: Applied Materials, Inc., Santa Clara, CA (US)

(72) Inventors: Kevin L. CUNNINGHAM, Mountain View, CA (US); Gregory Max MCDANIEL, San Jose, CA (US)

(21) Appl. No.: 15/415,208

(22) Filed: Jan. 25, 2017

Related U.S. Application Data
(60) Provisional application No. 62/294,222, filed on Feb. 11, 2016.

(51) Int. Cl. A61B 5/15; A61B 5/151; A61B 10/00

(52) U.S. Cl. CPC A61B 5/150969; A61B 10/0064; A61B 5/151

(57) ABSTRACT

Embodiments of the present disclosure generally relate to apparatuses for sampling bodily fluids and methods of fabrication thereof. The apparatus includes at least one adhesive patch, at least one bodily fluid port attached to the adhesive patch, and at least one bodily fluid storing capillary connected to the bodily fluid port and attached to the adhesive patch. The adhesive patch may be adhered to a subject's skin, the input port may then be connected to a source of bodily fluids and the one or more bodily fluid storing capillaries function to draw in the bodily fluid over time and store it for later analyzation. The one or more bodily fluid storing capillaries may be formed by imprint lithography on a plastic or glass substrate.

### CROSS-REFERENCE TO RELATED APPLICATIONS

This application claims benefit of U.S. Provisional Patent Application Ser. No. 62/294,222, filed on Feb. 11, 2016, which is herein incorporated by reference in its entirety.

### BACKGROUND

Field

Embodiments of the present disclosure generally relate to apparatuses for sampling bodily fluids and methods of fabrication thereof.

Description of the Related Art

Medical examinations are vitally important to maintaining a healthy global population. Various medical examinations are directed to testing bodily fluids, such as blood or sweat. Some bodily fluid tests must be taken over extended periods of time. Traditionally, an intravenous apparatus is attached to the subject. The subject must remain at the testing facility and connected to the bulky apparatus for an extended period of time. These apparatuses and methods are inconvenient to the subject.

Therefore, there is a need in the art for an improved apparatus for sampling bodily fluids and method of manufacture thereof.

### SUMMARY

Embodiments of the present disclosure generally relate to apparatuses for sampling bodily fluids and methods of fabrication thereof. The apparatus includes at least one adhesive patch, at least one bodily fluid port attached to the adhesive patch, and at least one bodily fluid storing capillary connected to the bodily fluid port and attached to the adhesive patch. The adhesive patch may be adhered to a subject's skin, the input port may then be connected to a source of bodily fluids and the one or more bodily fluid storing capillaries function to draw in the bodily fluid over time and store it for later analyzation. The one or more bodily fluid storing capillaries may be formed by imprint lithography on a plastic or glass substrate.

In one embodiment, an apparatus is disclosed. The apparatus includes an adhesive patch, a bodily fluid port attached to the adhesive patch and a single bodily fluid storing capillary connected to the bodily fluid port and attached to the adhesive patch. The single bodily fluid storing capillary comprises one or more curves.

In another embodiment, an apparatus is disclosed. The apparatus includes an adhesive patch, a bodily fluid port attached to the adhesive patch and a plurality of bodily fluid storing capillaries connected to the bodily fluid port and attached to the adhesive patch. Each of the plurality of bodily fluid storing capillaries is arranged around a circumference of the bodily fluid port.

In yet another embodiment, an apparatus is disclosed. The apparatus includes a plurality of adhesive patches and a plurality of bodily fluid ports. Each of the plurality of bodily fluid ports is attached to each of the plurality of adhesive patches.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 is a top down view of an apparatus according to one embodiment described herein.

FIG. 2 is a top down view of an apparatus according to another embodiment described herein.

FIG. 3 is a top down view of an apparatus according to another embodiment described herein.

FIG. 4 is a top down view of an apparatus according to another embodiment described herein.

FIG. 5 is a top down view of an apparatus according to another embodiment described herein.

FIG. 6A is a top down view of an apparatus according to yet another embodiment described herein.

FIG. 6B is a cross-sectional illustration of the apparatus of FIG. 6A.

FIGS. 7A-7B depict substrates at various stages of a method according to embodiments described herein.

FIG. 8 is a perspective view of a lithography system that may be used to perform embodiments disclosed herein.

To facilitate understanding, identical reference numerals have been used, wherever possible, to designate identical elements that are common to the Figures. Additionally, elements of one embodiment may be advantageously adapted for utilization in other embodiments described herein.

### DETAILED DESCRIPTION

Embodiments of the present disclosure generally relate to apparatuses for sampling bodily fluids and methods of fabrication thereof. The apparatus includes at least one adhesive patch, at least one bodily fluid port attached to the adhesive patch, and at least one bodily fluid storing capillary connected to the bodily fluid port and attached to the adhesive patch. The adhesive patch may be adhered to a subject's skin, the input port may then be connected to a source of bodily fluids and the one or more bodily fluid storing capillaries function to draw in the bodily fluid over time and store it for later analyzation. The one or more bodily fluid storing capillaries may be formed by imprint lithography on a plastic or glass substrate.

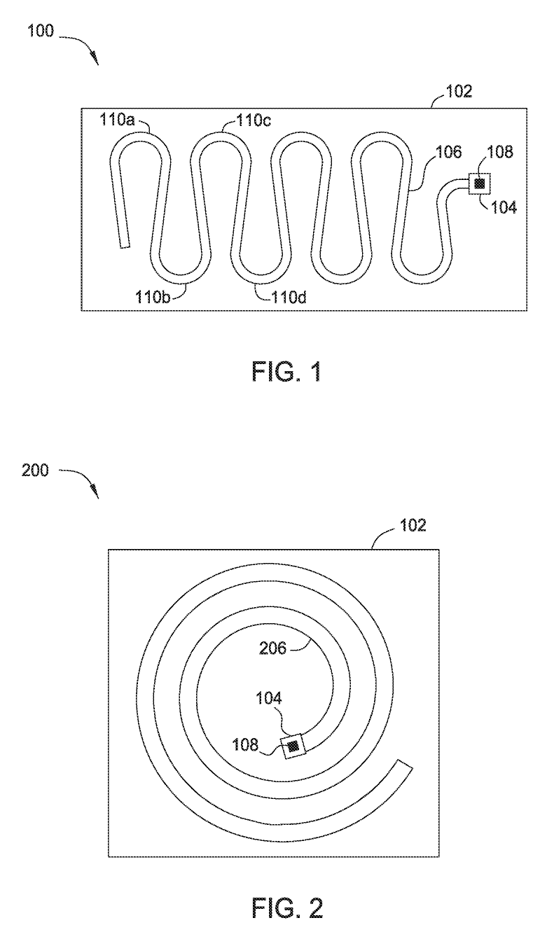

FIGS. 1-3 show top down views of an apparatus according to various embodiments described herein. Generally, each of the apparatuses 100, 200 and 300 include an adhesive patch 102, a bodily fluid port 104 and a bodily fluid storing capillary 106, 206 or 306, respectively. The bodily fluid port 104 and the bodily fluid storing capillary 106, 206 or 306 are attached to the adhesive patch 102. The bodily fluid storing capillary 106, 206 or 306 is connected to the bodily fluid port 104. The bodily fluid port 104 may further include a collector 108 for initially collecting from a source of bodily fluid and guiding the bodily fluid through the bodily fluid port 104. In one embodiment, the collector 108 may be a needle. In another embodiment the collector 108 may be a wicking material.

As shown in FIG. 1, the bodily fluid storing capillary 106 has a plurality of curves such that it has a serpentine shape. In use, the apparatus is attached to the subject's skin by the adhesive patch 102. A bodily fluid sample is introduced to the apparatus 100 at the bodily fluid port 104. In the event that the fluid to be sampled is blood, the apparatus 100 may further include the collector 108, specifically a needle. Once the bodily fluid enters the apparatus 100, the bodily fluid storing capillary 106 draws the bodily fluid into the length of the bodily fluid storing capillary 106 by capillary forces.

The apparatus 100 has eight curves; four are labeled (110a, 110b, 110c and 110d). Each of the curves may correspond to a different increment of time. For example, the sampling may take place over an 8-hour period. The bodily fluid taken initially may be held in the capillary up to the first curve 110a. The bodily fluid taken after the first hour may be held in the capillary between the first curve 110a and the second curve 110b. The bodily fluid taken during the second hour may be held in the capillary between the second curve 110b and the third curve 110c, and so on and so on until the 8-hour sampling period has ended.

As shown in FIG. 2, the bodily fluid storing capillary 206 has a plurality of curves such that it has a spiral shape. The curvature, dimensions, and surface treatment of the bodily fluid storing capillary 206 may be engineered to produce nearly constant capillary force such that the bodily fluid sample is drawn into the apparatus 200 at a nearly constant rate, or at a rate that has predictable variation.

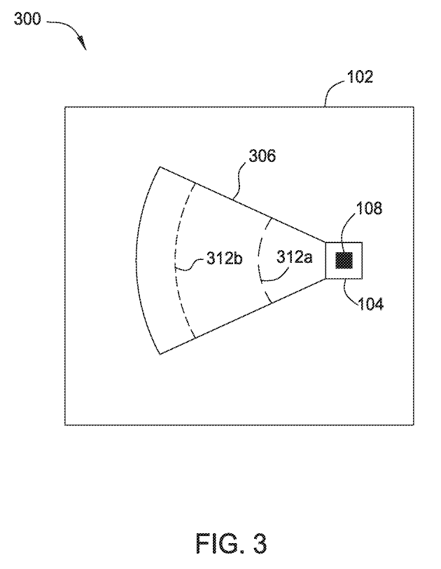

As shown in FIG. 3, the bodily fluid storing capillary 306 may have a single curve such that it has a wedge shape. The apparatus 300 further includes one or more porous membranes (two are shown) 312a, 312b. The one or more porous membranes 312a, 312b are used to separate the bodily fluid sample depending on cell size or components. Additionally, the one or more barrier films deposited on the surfaces of the structure are used to protect the bodily fluid sample from oxidation by ambient air. The first porous membrane 312a may have a first porosity and the second porous membrane 312b may have a second porosity. The pore size of the first porosity may be greater than the pore size of the second porosity such that larger cells cannot penetrate the second porosity of the second porous membrane 312b.

For example, white blood cells have a diameter of greater than 7 micrometers (pm). Red blood cells have a diameter of approximately 6.2 to 8.2 pm. However, red blood cells have an outer thickness of about 2 to 2.5 μm and an inner thickness of 0.8 to 1 μm. Plasma molecules may be even smaller. Therefore, the first porosity of the first porous membrane 312a can be configured such that only cells with a thickness of 6 μm or less can penetrate the first porous membrane 312a and the second porosity of the second porous membrane 312b can be configured such that only cells with a thickness of less than 0.8 μm can penetrate the second porous membrane 312b. In effect, the bodily fluid sample is separated into white blood cells, red blood cells and plasma with small plasma molecules like dissolved proteins (i.e.—serum albumins, antibodies, globulins, and fibrinogen),glucose, clotting factors, electrolytes, DNA, RNA, and hormones).

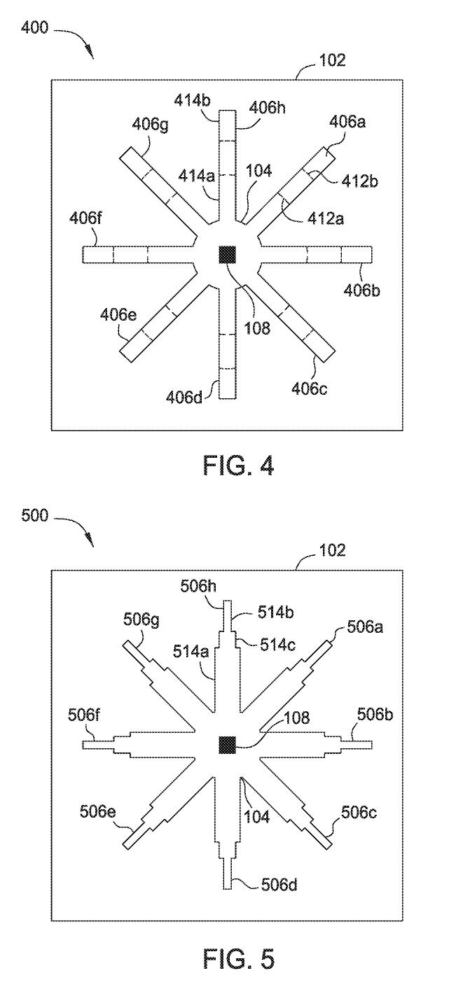

FIGS. 4-5 show top down views of an apparatus according to additional embodiments described herein. Generally, each of the apparatuses 400, 500 include an adhesive patch 102, a bodily fluid port 104 and plurality of bodily fluid storing capillaries (8 are shown) 406a-406h, 506a-506h, respectively. The bodily fluid port 104 and the bodily fluid storing capillaries 406a-406h, 506a-506h are attached to the adhesive patch 102. The bodily fluid storing capillaries 406a-406h, 506a-506h are connected to the bodily fluid port 104. The bodily fluid port 104 may further include a collector 108 for initially collecting from a source of bodily fluid and guiding the bodily fluid through the bodily fluid port 104. In one embodiment, the collector 108 may be a needle. In another embodiment the collector 108 may be a wicking material.

As shown in FIG. 4, the plurality of bodily fluid storing capillaries 406a-406h are arranged around, and extend from, the bodily fluid port 104. Each of the plurality of bodily fluid storing capillaries 406a-406h has a first end 414a and a second end 414b. The first volume at the first end 414a and the second volume at the second end 414b are equal. The apparatus 400 further includes one or more porous membranes (two are shown) 412a, 412b. The one or more porous membranes 412a, 412b are used to separate the bodily fluid sample depending on cell size or components. Additionally, the one or more porous membranes 412a, 412b are used to protect the bodily fluid sample from oxidation. The first porous membrane 412a may have a first porosity and the second porous membrane 412b may have a second porosity. The pore size of the first porosity may be greater than the pore size of the second porosity such that larger cells cannot penetrate the second porosity of the second porous membrane 412b.

Similar to the first porous membrane 312a and the second porous membrane 312b of FIG. 3, the first porosity of the first porous membrane 412a can be configured such that only cells with a thickness of 6 μm or less can penetrate the first porous membrane 412a and the second porosity of the second porous membrane 412b can be configured such that only cells with a thickness of less than 0.8 μm can penetrate the second porous membrane 412b. In effect, the bodily fluid sample is separated into white blood cells, red blood cells and plasma with small plasma molecules.

As shown in FIG. 5, the plurality of bodily fluid storing capillaries 506a-506h are arranged around, and extend from, the bodily fluid port 104. Each of the plurality of bodily fluid storing capillaries 506a-506h has a first end 514a and a second end 514b. The first volume at the first end 514a is greater than the second volume at the second end 514b. Each of the plurality of bodily fluid storing capillaries 506a-506h may further include an intermediate portion 514c between the first end 514a and the second end 514b. The intermediate portion 514c may have an intermediate volume. The varying volumes result in various restrictions such that the bodily fluid sample may be separated by characteristic sizes of the molecules. In other words, the geometry of each of the plurality of bodily fluid storing capillaries 506a-506h is tapered to reduce volume over the length of the capillary.

For example, the first volume at the first end 514a may store white blood cells and the second volume at the second end 514b may store smaller cells such as red blood cells and/or plasma with small plasma molecules. If the apparatus 500 includes an intermediate portion 514c, the first volume at the first end 514a may store white blood cells, the intermediate volume at the intermediate portion 514c may store red blood cells and the second volume at the second end 514b may store plasma with small plasma molecules.

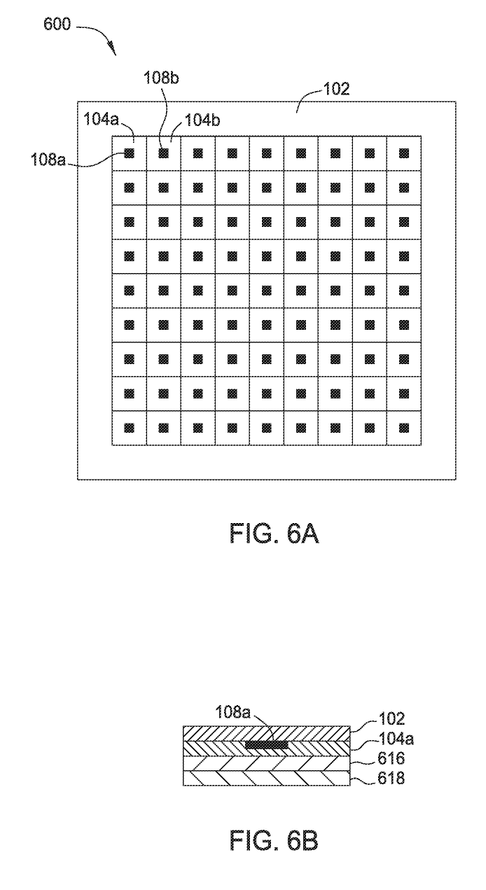

FIG. 6A is a top down view of an apparatus 600 according to yet another embodiment described herein. The apparatus 600 includes an adhesive patch 102 and a plurality of bodily fluid ports 104 arranged in an array. The plurality of bodily fluid ports 104 are attached to the adhesive patch 102. The apparatus 600 further includes a plurality of films (not labeled). The plurality of bodily fluid ports 104 are also attached the plurality of films. Each of the plurality of bodily fluid ports 104 may further include a collector 108 for initially collecting from a source of bodily fluid and guiding the bodily fluid through the bodily fluid port 104. In one embodiment, the collector 108 may be a needle. In another embodiment the collector 108 may be a wicking material. The apparatus 600 may further include a plurality of bodily fluid storing capillaries (not shown).

FIG. 6B is a cross-sectional view of a first bodily fluid port 104a of the plurality of bodily fluid ports 104. A first film 616 contacts the skin of the subject 618 such that the first bodily fluid port 104a is sandwiched between the adhesive patch 102 and the first film 616. The first bodily fluid port 104a includes a first collector 108a. The first film 616 may be made of a first material with a first water-solubility. In use, for example in sampling sweat, the first film 616 will dissolve after a first time period depending on the value of the first water-solubility. Once the first film 616 has dissolved, the first bodily fluid port 104a comes into contact with the subject's skin. The first collector 108a, in this example, a wicking material, also contacts the subject's skin and guides the bodily fluid sample into the first bodily fluid port 104a. As the sequence continues, a second film (not labeled), made of a second material with a second water-solubility will dissolve after a second time period depending on the value of the second water-solubility. Once the second film has dissolved, the second bodily fluid port 104b comes into contact with the subject's skin. The second collector 108b, also a wicking material, also contact's the subject's skin and guides the bodily fluid sample into the second bodily fluid port 104b. The sequence may repeat until each of the plurality of bodily fluid ports 104 has contacted the subject's skin and collected a portion of the bodily fluid sample.

While the aforementioned example contemplates that each of the plurality of films will have a different water-solubility, other properties of the film may change over the array such that each of the plurality of bodily fluid ports 104 are exposed to the subject's skin in sequence.

In additional embodiments, a subject may introduce a bodily fluid sample directly into the bodily fluid port 104. For example, the subject may introduce a single drop of blood into the bodily fluid port 104 at each hour of the sampling period.

After the bodily fluid has been sampled and is being stored in the apparatus for sampling bodily fluids, the bodily fluid sample may be removed from the device in several ways. For example, the bodily fluid sample may be removed by vacuum draw where the bodily fluid sample would be drawn out by a vacuum into a container. Alternatively, the bodily fluid sample may be removed by flushing the device with an additional fluid (gas or liquid fluid) to propel the sample out. The entire bodily fluid sample may be removed from the device at one time, or aliquots of the bodily fluid sample may be removed at various time periods over the course of the sampling. Additionally, the apparatus may be cut into several pieces before the bodily fluid sample is removed to ensure that the time-dependence of the sample is preserved.

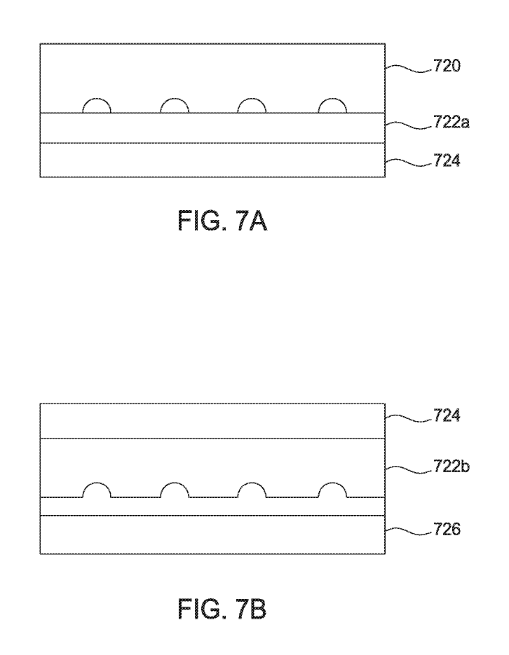

FIGS. 7A-7B depict substrates at various stages of a method according to embodiments described herein. Each of the embodiments described herein may be manufactured using imprint lithography. Specifically, the one or more bodily fluid ports and/or the one or more bodily fluid storing capillaries may be fabricated by imprint lithography, which is a method of fabricating nanometer scale patterns. The method includes depositing a resist material 722a on a first substrate 724. The resist material 722a would then be imprinted using a stamp 720. The patterned resist material 722b is then cured by ultra-violet (UV) light or heat and separated from the stamp 720. The patterned resist material 722b may then be adhered to a second substrate 726 or to another patterned layer. In one embodiment, the first and second substrates 724, 726 may be glass. In another embodiment, the first and second substrates 724, 726 may be plastic. For example, the first and second substrates 724, 726 may be polyethylene terephthalate (PET) or polyethylene naphthalate (PEN). After the shapes of the one or more bodily fluid ports and/or the one or more bodily fluid storing capillaries have been formed by imprint lithography, additional films may be deposited on the surface of the substrate. The additional films may include a water encapsulation layer, a porous membrane, hydrophilic layer or a hydrophobic layer. The surface may be further coated with molecules that have the property of spectively adhering to specific target molecules, cells, bacteria, viruses, or other organisms. An example of such a molecule would be an antibody that is developed to bind to molecules on the surface of specific types of cancer cells). This antibody would be attached to the surface of the bodily fluid port. As the bodily fluid flowed past the antibody molecules, the cancer cells would bind to and be immobilized by the antibody molecules. This would allow the user to isolate those cancer cells more easily.

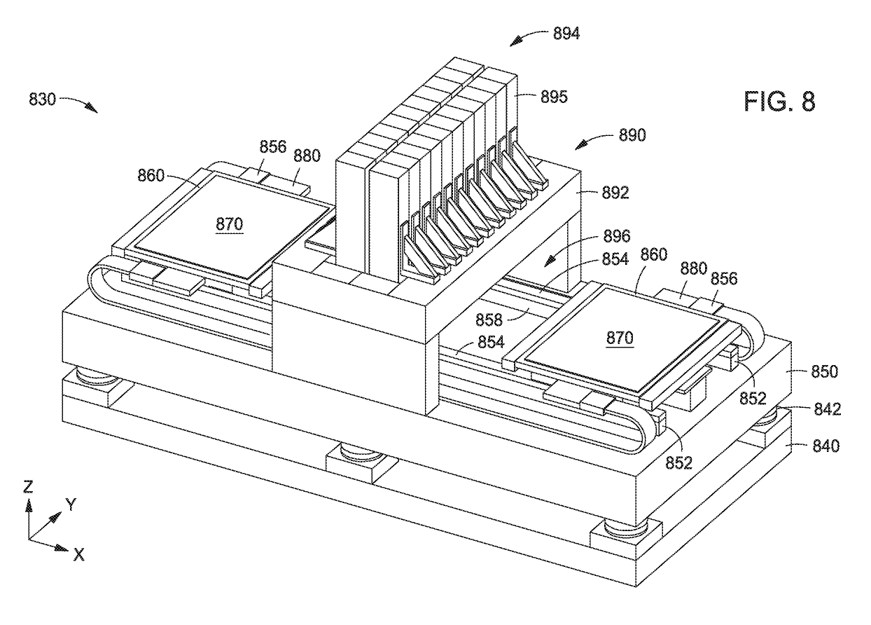

FIG. 8 is a perspective view of a lithography system 830 that may be used to perform the aforementioned processes. The system 830 includes a base frame 840, a slab 850, two or more stages 880, and a processing apparatus 890. The base frame 840 may rest on the floor of a fabrication facility and may support the slab 850. Passive air isolators 842 may be positioned between the base frame 840 and the slab 850. The slab 850 may be a monolithic piece of granite, and the two or more stages 880 may be disposed on the slab 850. A substrate 870 may be supported by each of the two or more stages 870. A plurality of holes (not shown) may be formed in the stage 860 for allowing a plurality of lift pins (not shown) to extend therethrough. The lift pins may rise to an extended position to receive the substrate 870, such as from a transfer robot (not shown). The transfer robot may position the substrate 870 on the lift pins, and the lift pins may thereafter gently lower the substrate 870 onto the stage 860.

As described in conjunction with FIGS. 7A-7B, the substrate 870 may, for example, be made of glass or plastic. In other embodiments, the substrate 870 may be made of other materials. The substrate 870 may have a photoresist layer formed thereon. A photoresist is sensitive to radiation and may be a positive photoresist or a negative photoresist, meaning that portions of the photoresist exposed to radiation will be respectively soluble or insoluble to photoresist developer applied to the photoresist after the pattern is written into the photoresist. The chemical composition of the photoresist determines whether the photoresist will be a positive photoresist or negative photoresist. For example, the photoresist may include at least one of diazonaphthoquinone, a phenol formaldehyde resin, poly(methyl methacrylate), poly(methyl glutarimide), and SU-8. In this manner, the pattern may be created on a surface of the substrate 870 to form the one or more bodily fluid storing capillaries.

The system 830 may further include a pair of supports 852 and a pair of tracks 854. The pair of supports 852 may be disposed on the slab 850, and the slab 850 and the pair of supports 852 may be a single piece of material. The pair of tracks 854 may be supported by the pair of the supports 852, and the two or more stages 860 may move along the tracks 854 in the X-direction. In one embodiment, the pair of tracks 854 is a pair of parallel magnetic channels. As shown, each track 854 of the pair of tracks 854 is linear. In other embodiments, the track 854 may have a non-linear shape. An encoder 856 may be coupled to each stage 860 in order to provide location information to a controller (not shown).

The processing apparatus 890 may include a support 892 and a processing unit 894. The support 892 may be disposed on the slab 850 and may include an opening 896 for the two or more stages 860 to pass under the processing unit 894. The processing unit 894 may be supported by the support 892. In one embodiment, the processing unit 894 is a pattern generator configured to expose a photoresist in a photolithography process. In some embodiments, the pattern generator may be configured to perform a maskless lithography process. The processing unit 894 may include a plurality of image projection systems disposed in a case 895. The processing apparatus 890 may be utilized to perform maskless direct patterning. During operation, one of the two or more stages 860 moves in the X-direction from a loading position to a processing position. The processing position may refer to one or more positions of the stage 860 as the stage 860 passes under the processing unit 894. During operation, the two or more stages 860 may be lifted by a plurality of air bearings and may move along the pair of tracks 854 from the loading position to the processing position. A plurality of vertical guide air bearings (not shown) may be coupled to each stage 860 and positioned adjacent an inner wall 858 of each support 852 in order to stabilize the movement of the stage 860. Each of the two or more stages 860 may also move in the Y-direction by moving along a track 880 for processing and/or indexing the substrate 870.

The lithography system 830 may be a commercially available lithography system from Applied Materials, Inc, of Santa Clara, Calif., or any suitable lithography system adapted for performing photolithography processes.

The use of imprint lithography of the manufacture of the one or more bodily fluid storing capillaries allows for production of capillaries, the dimensions and shapes of which are designed to draw in the bodily fluids by capillary forces, but are also small enough to prevent substantial mixing along the length of the capillary. The imprint lithography may by similar to that the imprint lithography described in U.S. Pat. No. 7,070,406 B2 or any other known method of imprint lithography.

Embodiments of the present disclosure allow for sampling of bodily fluids of at specific time periods over an extended period of time without inconveniencing the subject of the sampling. Furthermore, the described embodiments may be used to store the sampled bodily fluid, while largely preserving the time-dependent chemistry and biology of the sample, until the sample may be analyzed.

While the foregoing is directed to embodiments of the present disclosure, other and further embodiments of the disclosure may be devised without departing from the basic scope thereof, and the scope thereof is determined by the claims that follow.

What is claimed is:

1. An apparatus, comprising:
an adhesive patch;
a bodily fluid port attached to the adhesive patch; and
a single bodily fluid storing capillary connected to the bodily fluid port and attached to the adhesive patch, wherein the single bodily fluid storing capillary comprises a one or more curves.

2. The apparatus of claim 1, wherein the single bodily fluid storing capillary has a serpentine shape.

3. The apparatus of claim 1, wherein the single bodily fluid storing capillary has a spiral shape.

4. The apparatus of claim 1, further comprising a collector, wherein the collector is a needle connected to the bodily fluid port.

5. The apparatus of claim 1, further comprising a collector, wherein the collector is a wicking material connected to the bodily fluid port.

6. The apparatus of claim 1, wherein the single bodily fluid storing capillary has a wedge shape.

7. The apparatus of claim 6, wherein the single bodily fluid storing capillary further comprises a first porous membrane and a second porous membrane, wherein the first porous membrane has a first porosity and the second porous membrane has a second porosity, and wherein the first porosity is greater than the second porosity.

8. An apparatus, comprising:
an adhesive patch;
a bodily fluid port attached to the adhesive patch; and
a plurality of bodily fluid storing capillaries connected to the bodily fluid port and attached to the adhesive patch, wherein each of the plurality of bodily fluid storing capillaries is arranged around a circumference of the bodily fluid port.

9. The apparatus of claim 8, wherein each of the plurality of bodily fluid storing capillaries has a first volume at a first end and a second volume at a second end.

10. The apparatus of claim 9, wherein the first volume and the second volume are equal.

11. The apparatus of claim 10, wherein each of the plurality of bodily fluid capillaries further comprises a first porous membrane and a second porous membrane.

12. The apparatus of claim 9, wherein the first volume is greater than the second volume.

13. The apparatus of claim 12, wherein the first porous membrane has a first porosity and the second porous membrane has a second porosity.

14. The apparatus of claim 13, wherein the first porosity is greater than the second porosity.

15. An apparatus, comprising:
an adhesive patch; and
a plurality of bodily fluid ports, wherein each of the plurality of bodily fluid ports is attached to the adhesive patch, and wherein each of the plurality of bodily fluid ports is attached to each of a plurality of films.

16. The apparatus of claim 15, wherein a first film of the plurality of films is made of a first material having a first water-solubility.

17. The apparatus of claim 16, wherein a second film of the plurality of films is made of a second material having a second water-solubility.

18. The apparatus of claim 15, further comprising a plurality of collectors, wherein each of the plurality of collectors is a wicking material connected to each of the plurality of bodily fluid ports.

19. The apparatus of claim 15, further comprising a plurality of collectors, wherein each of the plurality of collectors is a needle connected to each of the plurality of bodily fluid ports.

20. The apparatus of claim 15, further comprising a plurality of bodily fluid storing capillaries, wherein each of the bodily fluid storing capillaries is connected to each of the plurality of bodily fluid ports and attached to each of the plurality of adhesive patches.
